(19) United States
(12) Patent Application Publication
(10) Pub. No.: US 2003/0175225 A1
(43) Pub. Date: Sep. 18, 2003

(54) Nail enamel composition exhibiting a color change dependent upon an observer's viewing angle

(76) Inventors: Tracey Leacock, Edison, NJ (US); Thomas R. Candia, Cedar Grove, NJ (US); Eric P. Wimmer, Princeton, NJ (US)

Correspondence Address: DUANE MORRIS LLP, 100 COLLEGE ROAD WEST, SUITE 100, PRINCETON, NJ, 08540-6604, US

(21) Appl. No.: 10/379,344

(22) Filed: Mar. 4, 2003

Related U.S. Application Data
(60) Provisional application No. 60/364,939, filed on Mar. 15, 2002.

(51) Int. Cl. A61K 7/04

(52) U.S. Cl. 424/61

(57) ABSTRACT

A nail enamel or nail polish composition includes aluminum platelets and silicon dioxide platelets, which silicon dioxide platelets are coated with a metal oxide to obtain an angle-dependent perceivable color shift in combination with a metallic or chrome-like background.

### PRIORITY FILING

This application claims the benefit of the earlier filing date, under 35 U.S.C. 119, of commonly assigned provisional patent application, Ser. No. 60/364,939 entitled, “Nail Enamel Composition Exhibiting a Color Change Dependent Upon an Observer's Viewing Angle”, filed on Mar. 15, 2002.

### FIELD OF INVENTION

The present invention relates generally to nail enamel compositions for external use on human or artificial nails; and more particularly to nail enamel compositions containing aluminum platelets, and silicon dioxide platelets which are coated with metal oxide.

### DESCRIPTION OF RELATED BACKGROUND ART

Various nail enamel compositions are known. New colors, clear formulations, and variations thereof are constantly being introduced by an extremely competitive cosmetics industry. Nail enamel compositions typically contain an organic solvent or mixture of solvents, film-forming agents, plasticizing ingredients, suspending agents, and colorants.

Most nail enamel or nail polish products contain nitrocellulose, either as a pigment dispersant or as a film forming binder. These nitrocellulose provide very good film forming properties when properly plasticized, and are relatively inexpensive materials. The tensile strength and the adhesive qualities of the enamel for the nail surface are generally inferior if nitrocellulose, or, alternatively, cellulose acetate butyrate is used without plasticizers.

Typical plasticizers used in nail enamel compositions include, for example, phthalates such as dibutyl phtbalate, citrates, including acetyl tributyl citrate and acetyl triethyl citrate, adipates, including dilsobutyl and dioctyl adipate, and/or camphor. Also included in a typical composition, as an adhesion promoter, is a polymeric component formed by the condensation polymerization of formaldehyde or other aldehyde, for example, an aromatic sulfonamide-aldehyde condensation resin such as ο-, ρ- toluene sulfonamide formaldehyde resin, or a polyester resin such as phthalic anhydride trimellitic anhydride/glycol copolymer.

Suspending agents protect the appearance of the product and prevent pigments, during the shelf life of the product, from floating, settling, separating, or striating the polish. In color nail enamel compositions, pigments, micas and/or dyes are incorporated and suspended in the various solvents and bases. Other known ingredients included in nail enamel formulations, for example, UV absorbers are added as needed, depending upon the particular application to which the nail enamel is intended.

All components of a nail enamel composition should be compatible. The nail enamel or polish composition should be a single phase, homogeneous composition that is free of grit and lumps and produces a smooth coating of uniform color and appearance that is clear and not cloudy or dull. Nail enamel compositions are also formulated to provide hardness, gloss, resistance to cracking, fading, chipping, peeling, and resistance to water, soaps, cleaning solutions and lotions after application and during wear. The nail enamel compositions should last for an extended period of time.

Based upon aesthetic considerations, a nail polish exhibiting color changes, which changes may vary depending upon the angle of view over a metallic background would provide an attractive nail polish for display purposes enhancing the beauty of the wearer's nails having the above properties.

There remains a need for improved nail enamel compositions exhibiting desired effects.

### SUMMARY OF THE INVENTION

Briefly described, the nail enamel composition includes at least one organic solvent, at least one film-forming substance, at least one plasticizer, at least one suspending agent, at least one adhesion promoter, a pigment based on silicon dioxide in the form of platelets, which platelets are coated with a metal oxide, and aluminum platelets. The metal oxide for coating the silicon dioxide particles is selected from TiO₂ (titanium dioxide), SnO (tin oxide), ferric oxide (Fe₂O₃), and combinations thereof.

The pigment is present in the composition in an amount ranging from about 0.5 to about 18.0 weight percent of the total composition, and has a particle size ranging from about 3 μm to about 60 μm (microns). The aluminum platelets are present in the composition in an amount ranging from about 0.03 to about 0.5 percent by weight of the total composition, and a particle size ranging from about 2 μm to about 26 μm.

The solvent is selected from the group consisting of butyl acetate, propyl acetate, ethyl acetate, isopropanol, butyl alcohol, and mixtures thereof. The film-forming substance is selected from the group consisting of: nitrocellulose, cellulose acetate butyrate, polyurethanes, acrylics, and mixtures thereof. The plasticizer is selected from the group consisting of dibutyl phthalate, diisobutyl adipate, acetyl tributyl citrate, acetyl triethylcitrate, camphor, and mixtures thereof. The suspending agent is selected from the group consisting of stearalkonium hectorite, stearalkonium bentonite, silica, montmorillonite clays, treated clays, and mixtures thereof. The adhesion promoter is selected from the group consisting of tosylamide epoxy resin, phthalic anhydride/trimellitic anhydride/glycol copolymer, tosylamide/formaldehyde resin, and adiptic acid/neopentyl glycol/trimellitic anhydride copolymer, and mixtures thereof.

The composition may further include an additional color or pigment component or other cosmetically acceptable component selected from the group consisting of D&C colorants, FD&C colorants, inorganic pigments, organic pigments, mica, guanine, and mixtures thereof.

### DETAILED DESCRIPTION OF THE INVENTION

The synthetically produced pigments employed in the present invention are based upon amorphous silicon dioxide (SiO₂) in the form of platelets or flakes. The pigments demonstrate a perceived color change that varies with a viewer's angle, and provide desired effects. The platelet-shaped synthetic pigments have a high lustre, opacity or transparency. The lustre is obtained by coating the silicon dioxide platelets with one or more thin, transparent, or semitransparent reflecting layers of metal oxides. The synthetic pigments are described in European Patent application serial number EP 0 608 388 B1, and EP 0960 911 A1, assigned to Merck Patent GmBH, the entire disclosures of which are hereby incorporated by reference. The pigments presently include Viola Fantasy (Product number F20-00) and Autumn Mystery (Product number T20-01), available from Merck KgaA in Darmstadt, Germany.

With regard to the Autumn Mystery pigment, silicon dioxide platelets are coated with ferric oxide (Fe₂O₃), and the color change ranges from red to gold and bronze to a green shimmer. With regard to the Viola Fantasy pigment, silicon dioxide platelets are coated with titanium dioxide (TiO₂), and the color change ranges from blue through green and silver, to a vivid purple. The synthetically produced pigments, in the form of platelets, lay flat on a natural or synthetic nail to provide a smooth, continuous surface. As more pigments in different shades or colors become available from the manufacturer, it is envisioned that the chemical nature of the pigments would operate with the composition of the present invention as defined by the appended claims by incorporating the silicon dioxide platelets with aluminum platelets in a nail enamel composition to attain a nail enamel which exhibits a color shift dependent upon an observation angle which provides an extremely attractive and unique nail polish.

The above described pigments, in combination with aluminum platelets provides unanticipated and aesthetic results of a color change over a metallic or chrome-like background. A preferred particle size for the aluminum platelets ranges from about 2 microns to about 26 microns. The aluminum platelets are available from Silberline Manufacturing Co., Inc., of Tamaqua, Pa., and are sold under the trademark STARBRITE®. Aluminum platelets are also available from Avery Dennison Corporation of Pasadena, Calif. The aluminum platelets, in combination with the synthetically produced pigments, produce a color travel or shift over a metallic background, which is aesthetically pleasing and unique.

A pigment comprising silicon dioxide in the form of platelets which are coated with a metal oxide is incorporated into the composition to provide a pearlized color change varying with the angle of view. A suitable metal oxide is selected from titanium oxide, tin oxide, ferric oxide, and combinations thereof. The angle-dependent color change is a perceived color shift, and is believed to be caused by the refraction of light due to the highly refractive metal oxides. The optical color effects thus appear to “travel” or “shift” as the angle of view changes.

The organic solvent selected for use in the present composition should be as inert as possible to the human or synthetic nail, and to other components of the nail enamel composition, without causing adverse reactions to the user or the composition. The solvent should also be capable of dissolving or dispersing the other components so that they readily flow onto the nail. The solvent must be able to evaporate from the nail in a matter of minutes at room temperature and pressure so that the film forming agents, plasticizers and other components dissolved therein solidify and adhere to the nail surface. Examples of preferred solvents include isopropanol, diacetone alcohol, ethyl acetate, butyl acetate, propyl acetate, and mixtures thereof. Toluene may also be used as a solvent. Effective amounts of the solvent component will generally lie in the range of about 30 wt % to about 90 wt % of the composition.

A film forming agent is necessary in order to provide the nail enamel with a consistency that results in the formulation of a uniform, stable film on the surface of the nail when the enamel is applied thereon. Nitrocellulose is the preferred film former and must be incorporated in the formulation in amounts that will cause the enamel to readily spread out about the surface of the nail yet remain viscous enough to not run or drip therefrom. However, it must also readily cling to and flow from the applicator brush as the enamel is applied from bottle to nail and this generally comprises from about 5.0 to 40 wt % of the total weight of the composition and preferably will be incorporated in amounts of from about 3 to about 30 wt %. Other suitable film formers include cellulose acetate butyrate, polyurethanes, and mixtures thereof. Acrylate copolymers may also be added as a secondary film forming agent.

Nail enamels must be durable to resist various physical and harsh household chemical attacks and must also be flexible, as the human nail is pliable and grows with time. To improve the tensile strength and adhesion characteristics of nail enamels, plasticizers and resins have generally been added to the nitrocellulose film forming composition. Dibutyl phthalate has been used as the plasticizer of choice for many years by providing excellent flexibility, elongation and tensile strength to nail polish formulations. Alternatively, adipates or citrates may be used as plasticizers, and include dioctyl adipate ((bis)-2-ethylhexyl adipate), diisobutyl adipate, acetyl tributyl citrate and acetyl triethyl citrate, and/or camphor. A plasticizer is incorporated in the nail enamel compositions of the present invention in amounts of from 0.1 to 15.0 weight percent (wt %) based on the total weight of the enamel composition.

A suspending agent should suspend the pigments in the nail enamel, and adjust the viscosity thereof to achieve desired flowability. Examples of preferred suspending agents for use in the present compositions include montmorillonite clays, and treated clays such as stearalkonium hectorite and bentonite. The amount of the suspending agent of course depends on the desired flow characteristics of the nail enamel. Amounts ranging from about 0.2 to about 2.0 wt % are generally satisfactory.

Adhesion promoters useful in the present invention include: phthalic anhydride/trimellitic anhydride/glycol copolymers and tosylamide formaldehyde resins, tosylamide epoxy resin, adipic acid/neopental glycol/trimellitic anhydride copolymer. An adhesion promoter is incorporated into the composition in amounts ranging from about 0.5 to about 20 weight percent of the total composition.

The nail enamel compositions of the present invention may include, in addition to the synthetically produced pigments, one or more additional pigment or other components. Suitable pigments include the inorganic and organic pigments that are cosmetically acceptable for use in cosmetic formulations. For example, particular examples of suitable pigments or FD&C colorants include titanium dioxide, D&C Red #6 Barium Lake, D&C Red #7 Calcium Lake, D&C Red #34 Calcium Lake, FD&C Yellow #5 Aluminum Lake, Ferric Ferrocyanide, Red Iron Oxide, Black Iron Oxide, Mica, Bismuth Oxychloride, Guanine, D&C Red #17, D&C Red #33, D&C Violet #2, D&C Yellow #11, FD&C Blue #1 and FD&C Green #3, and mixtures thereof. These pigments may also be surface treated for improved performance. See U.S. Pat. Nos. 5,133,966 to Khamis; 4,832,944 to Socci et al. and 5,174,996 to Weber et al., the disclosures of all of which are hereby incorporated herein by reference. The amounts of any particular ingredients comprising the additional pigment component will of course depend on the shade desired by the practitioner. Pearls or micas having a particle size of less than 150 microns may also be included to further enhance the desired effect. In general, the additional pigment component comprises about 0.01 to about 15.0 wt % of the composition.

Nail enamel compositions in accordance with the present invention may be manufactured by thoroughly and intimately mixing together all the components indicated above, in the amounts described above. Examples of satisfactory equipment and operation thereof is apparent to one of ordinary skill in the art. The mixing procedure for the nail enamel formulations of the present invention is as those generally known in the art wherein the film-forming component is first mixed into the solvent, followed by the plasticizer, and thereafter the remaining ingredients. All components are typically added while mixing the composition.

The present formulations are highly shelf stable and promote a superior and aesthetically pleasing nail enamel for a natural human or synthetic nails. The synthetically produced pigments coated with a metal oxide in combination with the aluminum platelets provide a unique color change effect on a metallic or mirror-like background. The following examples are provided for further describing the preferred formulations of the present invention. The examples are presented for illustrative purposes only and it is recognized that there may be minor changes and alterations made with respect to the ingredients and/or the amounts. It should be understood that to the extent any such changes do not materially affect the final composition or its functionality, they are considered to fall within the spirit and scope of the invention as recited in the appended claims. In all of the following examples, the values provided represent weight percentages based on 100% of the total weight of the composition.

### EXAMPLE 1

| | |
|---|---|
| Ethyl Acetate | 43.39 wt % |
| Butyl Acetate | 19.99 wt % |
| Nitrocellulose Dry | 8.79 wt % |
| Phthalic Anhydride/Trimellitic Anhydride/ | 6.54 wt % |
| Glycols Copolymer | |
| Color Stream T10-01 Viola Fantasy | 5.88 wt % |
| Sucrose Acetate Isobutyrate | 3.97 wt % |
| Isopropyl Alcohol | 3.96 wt % |
| Acrylates Copolymer | 2.95 wt % |
| Camphor | 1.27 wt % |
| Stearalkonium Hectorite | 1.09 wt % |
| Dibutyl Phthalate | 1.06 wt % |
| Diacetone Alcohol | 0.93 wt % |
| Benzophenone-1 | 0.13 wt % |
| Aluminum Powder | 0.03 wt % |
| Citric Acid | 0.02 wt % |
| TOTAL | 100.00 wt % |

### EXAMPLE 2

| | |
|---|---|
| Butyl Acetate | 25.51 wt % |
| Toluene | 21.00 wt % |
| Ethyl Acetate | 11.11 wt % |
| Nitrocellulose | 10.85 wt % |
| Tosylamide/Formaldehyde Resin | 9.20 wt % |
| Dibutyl Phthalate | 7.88 wt % |
| Color Stream T10-01 Viola Fantasy | 5.88 wt % |
| Isopropyl Alcohol | 4.89 wt % |
| Stearalkonium Hectorite | 1.32 wt % |
| Diacetone Alcohol | 1.13 wt % |
| Camphor | 1.00 wt % |
| Benzophenone-1 | 0.17 wt % |
| Aluminum Powder | 0.03 wt % |
| Citric Acid | 0.03 wt % |
| TOTAL | 100.00 wt % |

### EXAMPLE 3

| | |
|---|---|
| Ethyl Acetate | 64.33 wt % |
| Butyl Acetate | 14.32 wt % |
| Nitrocellulose | 7.03 wt % |
| Color Stream T10-01 Viola Fantasy | 5.88 wt % |
| Isopropyl Alcohol | 3.83 wt % |
| Diisobutyl Adipate | 2.00 wt % |
| Phthalic Anhydride/Trimellitic Anhydride/ | 1.28 wt % |
| Glycols Copolymer | |
| Stearalkonium Hectorite | 1.24 wt % |
| Citric Acid | 0.06 wt % |
| Aluminum Powder | 0.03 wt % |
| TOTAL | 100.00 wt % |

### EXAMPLE 4

| | |
|---|---|
| Ethyl Acetate | 44.57 wt % |
| Butyl Acetate | 19.42 wt % |
| Nitrocellulose | 8.54 wt % |
| Tosylamide/Epoxy Resin | 6.35 wt % |
| Color Stream T10-01 Viola Fantasy | 5.88 wt % |
| Sucrose Acetate Isobutyrate | 3.85 wt % |
| Isopropyl Alcohol | 3.85 wt % |
| Acrylates Copolymer | 2.87 wt % |
| Camphor | 1.24 wt % |
| Stearalkonium Hectorite | 1.05 wt % |
| Dibutyl Phthalate | 1.03 wt % |
| Diacetone Alcohol | 0.90 wt % |
| Aluminum Powder | 0.30 wt % |
| Benzophenone-1 | 0.13 wt % |
| Citric Acid | 0.02 wt % |
| TOTAL | 100.00 wt % |

### EXAMPLE 5

| | |
|---|---|
| Butyl Acetate | 26.77 wt % |
| Toluene | 22.05 wt % |
| Ethyl Acetate | 12.29 wt % |
| Nitrocellulose | 11.39 wt % |
| Tosylamide/Formaldehyde Resin | 9.65 wt % |
| Dibutyl Phthalate | 8.27 wt % |
| Isopropyl Alcohol | 5.14 wt % |
| Stearalkonium Hectorite | 1.39 wt % |
| Diacetone Alcohol | 1.19 wt % |
| Camphor | 1.05 wt % |
| Benzophenone-1 | 0.18 wt % |
| Color Stream T10-01 Viola Fantasy | 0.50 wt % |
| Aluminum Powder | 0.10 wt % |
| Citric Acid | 0.03 wt % |
| TOTAL | 100.00 wt % |

### EXAMPLE 6

| | |
|---|---|
| Ethyl Acetate | 39.62 wt % |
| Color Stream T10-01 Viola Fantasy | 18.00 wt % |
| Butyl Acetate | 16.54 wt % |
| Nitrocellulose | 7.23 wt % |
| Phthalic Anhydride/Trimellitic Anhydride/ | 5.40 wt % |
| Glycols Copolymer | |
| Isopropyl Alcohol | 3.28 wt % |
| Sucrose Acetate Isobutyrate | 3.22 wt % |
| Acrylates Copolymer | 2.39 wt % |
| Camphor | 1.05 wt % |
| Stearalkonium Hectorite | 0.89 wt % |
| Dibutyl Phthalate | 0.87 wt % |
| Diacetone Alcohol | 0.82 wt % |
| Aluminum Powder | 0.50 wt % |
| Benzophenone-1 | 0.11 wt % |
| D&C Red #6 Barium Lake | 0.06 wt % |
| Citric Acid | 0.02 wt % |
| TOTAL | 100.00 wt % |

### EXAMPLE 7

| | |
|---|---|
| Ethyl Acetate | 62.976 wt % |
| Butyl Acetate | 14.200 wt % |
| Nitrocellulose | 7.180 wt % |
| Color Stream T10-01 Viola Fantasy | 6.410 wt % |
| Isopropyl Alcohol | 3.870 wt % |
| Acetyl Tributyl Citrate | 1.920 wt % |
| Phthalic Anhydride/Trimellitic Anhydride/ | 1.520 wt % |
| Glycols Copolymer | |
| Stearalkonium Hectorite | 1.240 wt % |
| Dibutyl Phthalate | 0.180 wt % |
| Aluminum Powder | 0.100 wt % |
| Diacetone Alcohol | 0.100 wt % |
| Red Iron Oxide | 0.090 wt % |
| Ferric Ammonium Ferrocyanide | 0.060 wt % |
| Citric Acid | 0.060 wt % |
| Camphor | 0.040 wt % |
| D&C Red #6 Barium Lake | 0.030 wt % |
| D&C Red #7 Calcium Lake | 0.020 wt % |
| Titanium Dioxide | 0.001 wt % |
| Benzophenone-1 | 0.001 wt % |
| FD&C Yellow #5 Aluminum Lake | 0.001 wt % |
| Black Iron Oxide | 0.001 wt % |
| TOTAL | 100.000 wt % |

### EXAMPLE 8

| | |
|---|---|
| Ethyl Acetate | 63.199 wt % |
| Butyl Acetate | 14.990 wt % |
| Nitrocellulose | 7.160 wt % |
| Color Stream T10-01 Viola Fantasy | 5.300 wt % |
| Isopropyl Alcohol | 3.880 wt % |
| Diisobutyl Adipate | 2.050 wt % |
| Phthalic Anhydride/Trimellitic Anhydride/ | 1.470 wt % |
| Glycols Copolymer | |
| Stearalkonium Hectorite | 1.240 wt % |
| FD&C Blue #1 Aluminum Lake | 0.200 wt % |
| Sucrose Acetate Isobutyrate | 0.140 wt % |
| Red Iron Oxide | 0.090 wt % |
| Diacetone Alcohol | 0.070 wt % |
| Dibutyl Phthalate | 0.070 wt % |
| Citric Acid | 0.060 wt % |
| Acrylates Copolymer | 0.040 wt % |
| Sucrose Benzoate | 0.030 wt % |
| Camphor | 0.030 wt % |
| D&C Red #6 Barium Lake | 0.030 wt % |
| Aluminum Powder | 0.030 wt % |
| Benzophenone-1 | 0.001 wt % |
| TOTAL | 100.000 wt % |

### EXAMPLE 9

| | |
|---|---|
| Ethyl Acetate | 43.05 wt % |
| Butyl Acetate | 20.24 wt % |
| Nitrocellulose | 8.79 wt % |
| Adipic Acid/Neopentyl Glycol/Trimellitic | 6.52 wt % |
| Anhydride Copolymer | |
| Color Stream T10-01 Viola Fantasy | 5.44 wt % |
| Sucrose Acetate Isobutyrate | 4.01 wt % |
| Isopropyl Alcohol | 3.97 wt % |
| Acrylates Copolymer | 2.90 wt % |
| Camphor | 1.25 wt % |
| Stearalkonium Hectorite | 1.08 wt % |
| Dibutyl Phthalate | 1.03 wt % |
| Diacetone Alcohol | 0.96 wt % |
| FD&C Blue #1 Aluminum Lake | 0.20 wt % |
| Benzophenone-1 | 0.13 wt % |
| Bismuth Oxychloride | 0.13 wt % |
| Diisobutyl Adipate | 0.12 wt % |
| Aluminum Powder | 0.07 wt % |
| Sucrose Benzoate | 0.04 wt % |
| D&C Red #6 Barium Lake | 0.03 wt % |
| Citric Acid | 0.02 wt % |
| D&C Yellow #10 Aluminum Lake | 0.02 wt % |
| TOTAL | 100.00 wt % |

What is claimed is:

1. A nail enamel composition comprising:
a pigment, comprising silicon dioxide in the form of platelets coated with a metal oxide.

2. The composition according to claim 1, wherein the metal oxide is selected from the group consisting of TiO₂ (titanium dioxide); SnO (tin oxide); ferric oxide (Fe₂O₃); and combinations thereof.

3. The composition according to claim 2, wherein said pigment is present in the composition in an amount ranging from about 0.5 to about 18 weight percent of the total composition.

4. The composition according to claim 1, further including aluminum platelets.

5. The composition according to claim 3, wherein said pigment has a particle size ranging from about 3 μm to about 60 μm.

6. The composition according to claim 4, wherein said aluminum platelets have a particle size ranging from about 2 μm to about 26 μm.

7. The composition of claim 2, further comprising: a solvent selected from the group consisting of butyl acetate, propyl acetate, ethyl acetate, isopropanol, butyl alcohol, and mixtures thereof.

8. The composition of claim 2, further comprising: a film-forming substance selected from the group consisting of: nitrocellulose, cellulose acetate butyrate, polyurethanes, acrylics, and mixtures thereof.

9. The composition according to claim 2, further comprising: a plasticizer selected from the group consisting of dibutyl phthalate, diisobutyl adipate, acetyl tributyl citrate, acetyl triethyl citrate, camphor, and mixtures thereof.

10. The composition of claim 2, further comprising: a suspending agent selected from the group consisting of stearalkonium hectorite, stearalkonium bentonite, silica, montmorillonite clays, treated clays, and mixtures thereof.

11. The composition of claim 2, further comprising a coloring agent selected from the group consisting of D&C colorants, FD&C colorants, inorganic pigments, organic pigments, mica, guanine, and mixtures thereof.

12. A nail enamel composition comprising:
at least one organic solvent;
at least one film-forming substance;
at least one plasticizer selected from the group consisting of: dibutyl phthalate, diusobutyl adipate, acetyl tributyl citrate, acetyl triethyl citrate, camphor, and mixtures thereof;
at least one suspending agent;
at least one adhesion promoter selected from the group consisting of tosylamide epoxy resin, phthalic anhydride/trimellitic anhydride/glycol copolymer, tosylamide/formaldehyde resin, and adiptic acid/neopentyl glycol/trimellitic anhydride copolymer, and mixtures thereof;
a pigment, comprising silicon dioxide in the form of platelets, which platelets are coated with a metal oxide.

13. The composition according to claim 12, wherein the metal oxide is selected from the group consisting of TiO₂ (titanium dioxide); SnO (tin oxide); ferric oxide (Fe₂O₃); and combinations thereof.

14. The composition according to claim 13, wherein said solvent is selected from the group consisting of butyl acetate, propyl acetate, ethyl acetate, isopropanol, butyl alcohol, and mixtures thereof.

15. The composition according to claim 13, wherein said film-forming agent is selected from the group consisting of: nitrocellulose, cellulose acetate butyrate, polyurethanes, acrylics, and mixtures thereof.

16. The composition according to claim 13, wherein said suspending agent is selected from the group consisting of stearalkonium hectorite, stearalkonium bentonite, silica, montmorillonite clays, treated clays, and mixtures thereof.

17. The composition according to claim 13, further comprising a coloring agent selected from the group consisting of D&C colorants, FD&C colorants, inorganic pigments, organic pigments, mica, guanine, and mixtures thereof.

18. A nail polish composition, comprising:
a pigment suspended in a nail enamel composition and operative to provide a color shift depending upon the angle of view.

19. The nail polish composition according to claim 18 further including aluminum platelets.

20. The composition according to claim 19 wherein said aluminum platelets have a particle size ranging from about 2 μm to about 26 μm.
